(19) United States
(12) Patent Application Publication
(10) Pub. No.: US 2016/0341241 A1
(43) Pub. Date: Nov. 24, 2016

(54) ENDOSCOPE

(71) Applicant: OLYMPUS CORPORATION, Tokyo (JP)

(72) Inventor: Yoichi HOSAKA, Iruma-shi (JP)

(73) Assignee: OLYMPUS CORPORATION, Tokyo (JP)

(21) Appl. No.: 15/228,091

(22) Filed: Aug. 4, 2016

Related U.S. Application Data
(63) Continuation of application No. PCT/JP2014/069670, filed on Jul. 25, 2014.

(30) Foreign Application Priority Data
Feb. 7, 2014 (JP) 2014-022478

(51) Int. Cl. F16C 1/10; F16C 1/14; F16C 1/12; A61B 1/005

(52) U.S. Cl. CPC F16C 1/106; A61B 1/0052; F16C 1/14; F16C 1/12

(57) ABSTRACT

An endoscope includes an insertion section including a bending portion, a bending wire inserted through the insertion section and configured to bend the bending portion, a protruding portion of a suspension frame configured to pull a proximal end portion of the bending wire, and a screw that is fixed to the protruding portion, includes a hole through which the bending wire is inserted, and is configured to latch the proximal end portion of the bending wire in a state where the bending wire is rotatable in the hole around an axis of the bending wire.

### CROSS REFERENCE TO RELATED APPLICATION

This application is a continuation application of PCT/JP2014/069670 filed on Jul. 5, 2014 and claims benefit of Japanese Application No. 2014-022478 filed in Japan on Feb. 7, 2014, the entire contents of which are incorporated herein by this reference.

### BACKGROUND OF THE INVENTION

1. Field of the Invention

The present invention relates to an endoscope, and more particularly, to an endoscope having a bending mechanism using a bending wire.

2. Description of the Related Art

Conventionally, endoscopes are widely used in a medical field and an industrial field. An endoscope has an elongated insertion section, and a bending portion is provided at a distal end portion of the insertion section. An operator of the endoscope is allowed to bend the bending portion by operating a bending operation section of an operation section connected to a proximal end of the insertion section. For example, the bending portion is configured to be able to bend in four directions of up, down, left and right, and the operator of the endoscope may bend the bending portion in a desired direction by operating the bending operation section. As a result, the operator may smoothly insert the insertion section into an examination target, and position an examination part at a center of an endoscopic image to make observations of the same.

A plurality of bending wires are inserted through the insertion section, and distal ends of respective bending wires are fixed to one bending piece of the bending portion, and proximal ends of respective bending wires are fixed to the bending operation section. When a movement of the bending operation section is transmitted to the bending portion, the bending portion is bent by the plurality of bending wires.

To transmit a movement of the bending operation section to the bending portion without delay, each bending wire has to be taut between the bending operation section and the bending portion when the bending portion is straight without being bent. Accordingly, at the time of manufacturing of the endoscope, tension of each bending wire is adjusted so that each bending wire becomes taut between the bending operation section and the bending portion.

To adjust the tension, a configuration where a wire joining portion for adjusting the tension of the bending wire is provided somewhere around a middle of each bending wire is proposed, as disclosed in Japanese Patent Application Laid-Open Publication No. 2011-206187 and Japanese Patent Application Laid-Open Publication No. 2001-37706.

The wire joining portion changes a length of each bending wire so as to place each bending wire in a taut state, by screwing together an adjustment screw serving as a male screw member and a joining/retention member having a female screw portion and by adjusting the amount of screwing of the adjustment screw into the joining/retention member.

### SUMMARY OF THE INVENTION

An endoscope according to an aspect of the present invention includes an insertion section including a bending portion, a bending wire inserted through the insertion section and configured to bend the bending portion, a pulling portion configured to pull a proximal end portion of the bending wire, and a latch member including a screw portion, fixed to the pulling portion by being screwed, and including a hole through which the bending wire is inserted, the latch member being configured to latch the proximal end portion of the bending wire in a state where the bending wire is rotatable in the hole around an axis of the bending wire.

### BRIEF DESCRIPTION OF THE DRAWINGS

FIG. 1 is a perspective view of an endoscope system provided with an endoscope according to an embodiment of the present invention;

FIG. 2 is a cross-sectional view of an operation section 6, according to the embodiment of the present invention, for describing a wire pulling mechanism for pulling a plurality of bending wires;

FIG. 3 is a partial perspective view of the wire pulling mechanism according to the embodiment of the present invention;

FIG. 4 is across-sectional view of a retention portion 35 and a latch portion 34 of a protruding portion 32 according to the embodiment of the present invention;

FIG. 5 is a schematic view for describing a method, according to the embodiment of the present invention, for adjusting tension of a bending wire 33 so as to make each bending wire 33 taut between a wire pulling mechanism 21 and a bending portion 12 when the bending portion 12 is straight without being bent;

FIG. 6 is a cross-sectional view of a retention portion 35 and a latch portion 34 of a protruding portion 32 provided with a plurality of spacers 61, according to a modification of the embodiment of the present invention; and

FIG. 7 is a perspective view of the spacer 61 according to the modification of the embodiment of the present invention.

### DETAILED DESCRIPTION OF THE PREFERRED EMBODIMENT

Hereinafter, an embodiment of the present invention will be described with reference to the drawings.

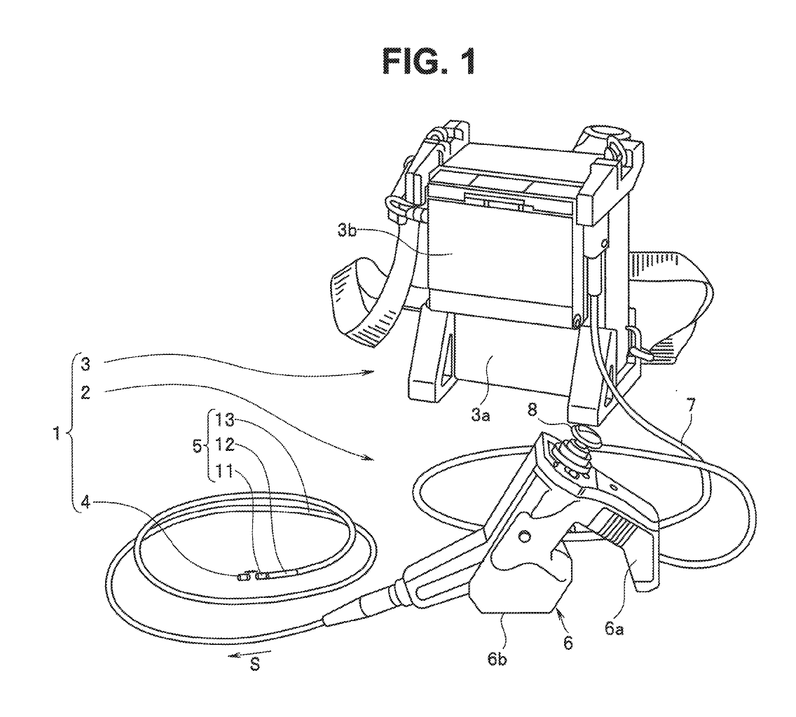

FIG. 1 is a perspective view of an endoscope system provided with an endoscope according to an embodiment of the present invention.

As shown in FIG. 1, an endoscope system 1 is configured mainly by including an endoscope 2 including a bending mechanism which uses bending wires, a device main body 3 connected to the endoscope 2, and an adapter 4.

The endoscope 2 is configured mainly by including an elongated flexible insertion section 5, an operation section 6, with a grasping portion 6a, connected to a proximal end of the insertion section 5 in an insertion direction S, and a universal cord 7 extending from the grasping portion 6a of the operation section 6.

A distal end portion 11 to which the adapter 4 may be freely attached or detached, a bending portion 12 which can be freely bent in a plurality of directions (in this case, four directions of up, down, left and right) by operation of a joystick 8 provided in the operation section 6, and a long flexible tube portion 13 formed by a flexible member are continuously provided in the insertion section 5, in this order from a distal end side of the insertion section 5, and a proximal end of the flexible tube portion 13 is connected to the operation section 6. A plurality of bending wires 33, described later, for bending the bending portion are inserted through the flexible tube portion 13 of the insertion section 5. A wire pulling mechanism for pulling or slackening the plurality of bending wires 33 according to movement of the joystick 8 is provided inside a housing 6b of the operation section 6.

Note that, in addition to the joystick 8, the operation section 6 is provided with various switches and the like, not shown, such as for instructing an image pickup device (not shown) provided inside the distal end portion 11 to perform an image pickup operation.

The device main body 3 is box-shaped, for example, and has a monitor 3b for displaying an endoscopic image, which is captured by the image pickup device (not shown) of the endoscope 2, fixed to an outer housing 3a made of magnesium die cast, for example, in a manner capable of freely opening or closing with respect to the outer housing 3a. Note that the monitor 3b may be freely detached/attached from/to the outer housing 3a, or may be fixed to the outer housing 3a with a monitor surface being constantly exposed.

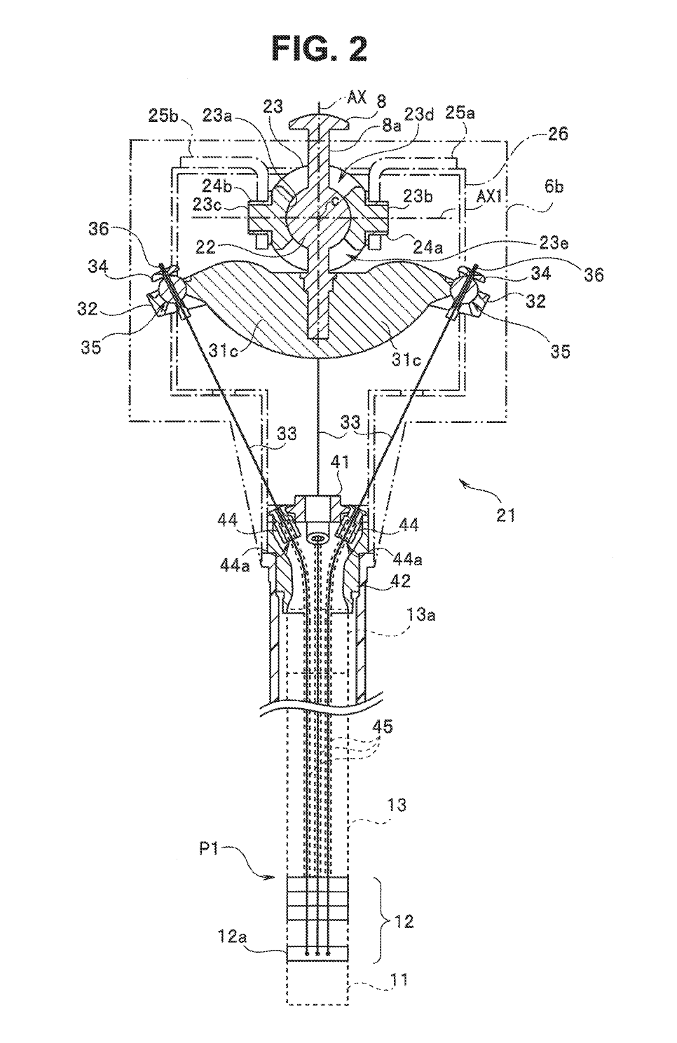

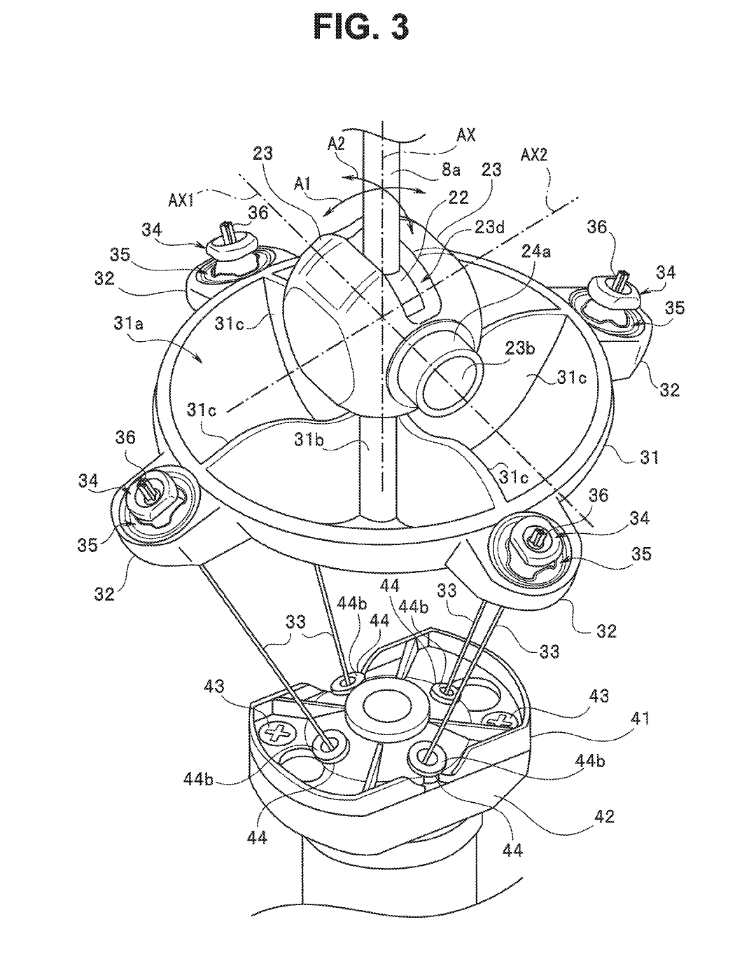

FIG. 2 is a cross-sectional view of the operation section 6, for describing the wire pulling mechanism for pulling the plurality of bending wires. FIG. 3 is a partial perspective view of the wire pulling mechanism. FIG. 2 shows a cross-section along an axial direction of two bending wires.

As shown in FIG. 2, a wire pulling mechanism 21 is provided inside the housing 6b (shown by a two-dot chain line) of the operation section 6.

The wire pulling mechanism 21 includes the metal joystick 8. A shaft portion 8a of the joystick 8 has a spherical portion 22. The spherical portion 22 is retained inside a concave portion 23a of a resin retention portion 23, in a manner capable of rotating around a center c of the spherical portion 22. Therefore, the concave portion 23a has a spherical shape.

The retention portion 23 includes two columnar shaft portions 23b and 23c. Two axes of the two shaft portions 23b and 23c are on one straight line along an axis AX1. The two shaft portions 23b and 23c are supported by respective bearing portions 24a and 24b, and the retention portion 23 is rotatable around the common axis AX1 of the two shaft portions 23b and 23c. The two bearing portions 24a and 24b are supported by respective support members 25a and 25b. The two support members 25a and 25b are fixed to a frame member 26 inside the housing 6b. Accordingly, the shaft portion 8a of the joystick 8 is capable of rotating around the axis AX1, and of being tilted in a direction shown by an arrow A1 in FIG. 3.

Furthermore, the retention portion 23 includes two long holes 23d and 23e. The holes 23d and 23e have a long shape extending along the direction of the axis AX1. The two holes 23d and 23e are formed point-symmetrical with respect to the center c of the spherical portion 22. The shaft portion 8a of the joystick 8 extends from the spherical portion 22 through the two holes 23d and 23e. Axes AX1 and AX2 intersect with an axis AX of the shaft portion 8a of the joystick 8 at the center c of the spherical portion 22, and the axes AX1 and AX2 are both orthogonal to the axis AX. Accordingly, the shaft portion 8a of the joystick 8 is capable of rotating around the axis AX2, which is orthogonal to the axis AX1, and of being tilted in a direction shown by an arrow A2 in FIG. 3.

As described above, the shaft portion 8a of the joystick 8 is capable of being tilted in a desired direction around the center c of the spherical portion 22.

A proximal end portion of the shaft portion 8a of the joystick 8 is fixed to a suspension frame 31. More specifically, the suspension frame 31 is bowl-shaped with a concave portion 31a on an upper side. The suspension frame 31 includes, at a center of a bottom portion of the concave portion 31a, a columnar extending portion 31b extending from the bottom portion of the concave portion 31a toward a center of a circular opening. The proximal end portion of the shaft portion 8a of the joystick 8 is fixed by being fitted inside the extending portion 31b of the suspension frame 31.

Note that the suspension frame 31 is combined with the joystick 8, and the joystick 8 is retained by the retention portion 23.

The suspension frame 31 includes, inside the concave portion 31a, four wall portions 31c extending in four directions from the extending portion 31b. When seen from an upper side along an axial direction of the columnar extending portion 31b, the four wall portions 31c are formed around the extending portion 31b with intervals of 90 degrees.

The suspension frame 31 includes four protruding portions 32 on an outer circumferential side of the concave portion 31a, on opposite sides of the respective wall portions 31c. Each protruding portion 32 includes a retention portion 35 for retaining a latch portion 34 described later. The latch portion 34 pulls the metal bending wire 33 when the bending wire 33 is latched to the latch portion 34. That is, the suspension frame 31 and the four protruding portions 32 configure a pulling portion for pulling proximal end portions of the four bending wires 33.

Four bending wires 33 are provided so as to bend the bending portion in an upward direction, which is a first direction, a downward direction, which is a second direction opposite the upward direction, a left direction, which is a third direction orthogonal to the upward direction, and a right direction, which is a fourth direction opposite the left direction. Moreover, the suspension frame 31 and the four protruding portions 32 move in such a way that, when one of a first pair of bending wires for bending in the upward/downward directions is pulled, the other of the pair of bending wires is slackened, and when one of a second pair of bending wires for bending in the left/right directions is pulled, the other of the pair of bending wires is slackened.

Note that the bending portion 12 in this case bends in four directions of up, down, left and right, but the bending portion 12 may alternatively be bent in two directions of up and down, or left and right. In the case of bending in two directions, two bending wires 33 are inserted through the insertion section 5.

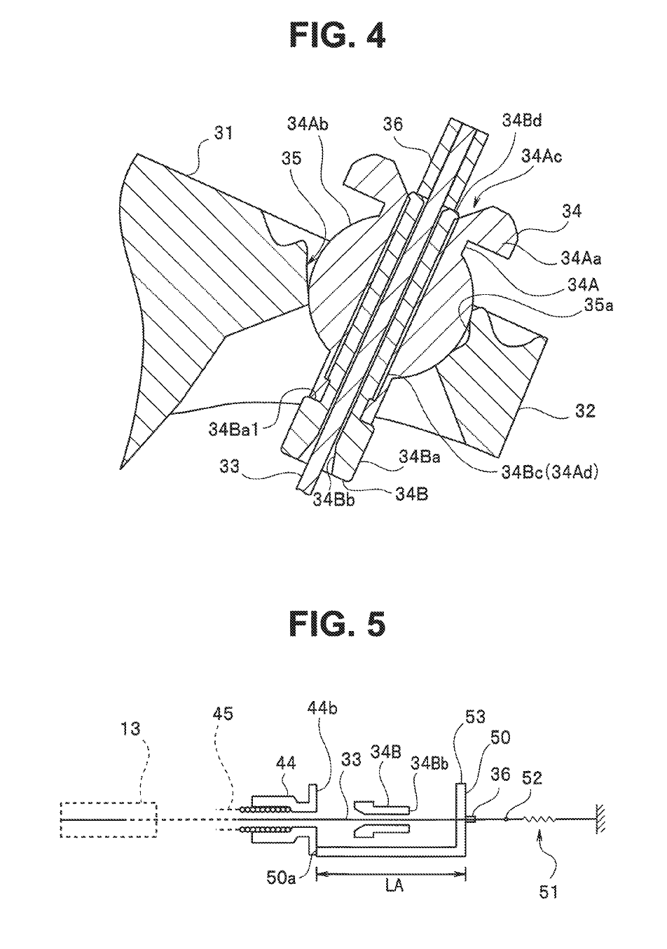

FIG. 4 is a cross-sectional view of the retention portion 35 and the latch portion 34 of the protruding portion 32. FIG. 4 shows cross-sections of the retention portion 35 and the latch portion 34 of the protruding portion 32, along a plane parallel to a surface of the wall portion 31c.

The latch portion 34 is configured from a cylindrical member 34A having an outward flange 34Aa at a distal end, and a cylindrical screw 34B. A spherical portion 34Ab is formed at a center portion of the metal cylindrical member 34A. A concave portion 34Ac is formed at a distal end side of the cylindrical member 34A.

The retention portion 35 includes a concave portion 35a into which the spherical portion 34Ab of the cylindrical member 34A is to be fitted.

A grasping portion 34Ba having a shape of an outward flange is provided at a distal end side of the metal screw 34B. The screw 34B is a cylindrical screw, and a screw portion 34Bc is threaded on an outer circumferential portion of the screw 34B, from the grasping portion 34Ba toward a proximal end side. A screw portion 34Ad is threaded on an inner circumferential surface of a hole of the cylindrical member 34A.

That is, the cylindrical member 34A includes the screw portion 34Ad, which is a female screw portion, on the inner circumferential surface of the hole, and the screw 34B is a screw member having the screw portion 34Bc, which is a male screw portion, formed on the outer circumferential surface. Accordingly, a worker may fix the screw 34B in the cylindrical member 34A by screwing together the screw portions 34Ad and 34Bc and rotating the screw 34B around the axis by grasping the grasping portion 34Ba of the screw 34B to thereby screw the screw 34B into the hole of the cylindrical member 34A until a proximal end surface 34Ba1 of the grasping portion 34Ba abuts a distal end portion of the cylindrical member 34A.

The bending wire 33 is inserted through a hole 34Bb of the cylindrical screw 34B in a loosely fitted manner. The proximal end portion of the bending wire 33 is crimped by using a metal crimping member 36. The maximum outer diameter of the crimping member 36 after being crimped at the proximal end portion of the bending wire 33 is larger than an inner diameter of the hole 34Bb of the screw 34B. Accordingly, the proximal end portion of the bending wire 33 is prevented from being pulled out from the hole 34Bb of the screw 34B in a distal end direction of the bending wire 33, by the crimping member 36 abutting a proximal end surface 34Bd of the screw 34B.

That is, the crimping member 36 forms a proximal end portion attachment member which is attached and fixed to the proximal end portion of the bending wire 33. Moreover, when the crimping member 36 abuts the screw 34B, the proximal end portion of the bending wire 33 is latched to the screw 34B.

Referring back to FIGS. 2 and 3, a fixing member 41 is provided in the distal end portion of the operation section 6. The fixing member 41 is fixed, by fastening of a screw 43, to a support member 42 which is fixed to a frame member 26 inside the housing 6b of the operation section 6.

Four circular cylindrical metal fittings 44 are provided in the fixing member 41. The four bending wires 33 extending from an insertion section rear end portion 13a (shown by a dotted line) of the insertion section 5 are inserted through holes of respective metal fittings 44. The four metal fittings 44 are fixed to the fixing member 41 with proximal end portions of the four metal fittings 44 separated from one another, and each bending wire 33 extends straight to the protruding portion 32 from each metal fitting 44.

A concave portion 44a is formed at a distal end side (the insertion section 5 side) of each circular cylindrical metal fitting 44. A proximal end portion of a coil pipe 45 (shown by a dotted line) through which the bending wire 33 is inserted is fitted in each concave portion 44a. Each coil pipe 45 is a flexible compression coil, and is not reduced in length even when compressed in an axial direction.

Four coil pipes 45 are inserted through the flexible tube portion 13, and distal ends of the coil pipes 45 are fixed to a distal end member of the flexible tube portion 13, at a predetermined position P1 on a proximal end side of the bending portion 12. Also, distal ends of the bending wires 33 inserted through the four coil pipes 45 are fixed to a bending piece 12a at a most distal end of the bending portion 12, with intervals of 90 degrees around the axis of the insertion section 5.

### (Adjustment of Tension of Bending Wire)

Next, adjustment of tension of the four bending wires 33 of the endoscope described above will be described. FIG. 5 is a schematic view for describing a method for adjusting tension of the bending wires 33 so as to make each bending wire 33 taut between the wire pulling mechanism 21 and the bending portion 12 when the bending portion 12 is straight without being bent. Only one bending wire 33 is shown in FIG. 5.

First, because distal ends of the four bending wires 33 are fixed to the most distal end bending piece 12a, the proximal end of each bending wire 33 is passed through the coil pipe 45 and through the hole of the metal fitting 44 provided in the fixing member 41, and is inserted through the hole 34Bb of the screw 34B. The proximal end portions of the bending wires 33 are drawn out from proximal end sides of the holes 34Bb of the screws 34B, and a distal end portion of each bending wire 33 which has been drawn out is inserted through the hole of the circular cylindrical crimping member 36 which is not yet crimped.

In this state, the distal end of the bending wire 33 drawn out from the screw 34B is pulled by a weight or the like, and predetermined tension is applied to the bending wire 33. FIG. 5 shows that the proximal end of the bending wire 33 is connected to a spring end 52 of a spring 51 or the like, and that predetermined tension is applied to the bending wire 33.

In this state, the crimping member 36 is crimped by using a wire length adjustment member 53, such as a jig, such that a distance from a distal end surface of the crimping member 36 to a proximal end surface 44b of the metal fitting 44 is a design dimension LA. A proximal end surface 50 and a distal end surface 50a of the wire length adjustment member 53 are in contact with the distal end surface of the crimping member 36 and the proximal end surface 44b of the metal fitting 44, respectively, in such a way that the distance from the distal end surface of the crimping member 36 to the proximal end surface 44b of the metal fitting 44 is LA. An outer diameter dimension of the crimping member 36 after crimping is set to be smaller than a screw outer diameter of the screw 34B. Four wires are set in this manner.

The bending wire 33 on the proximal end side of the crimping member 36 is cut in this state. The screw 34B is screwed into a deep end of the hole of the cylindrical member 34A, and is fixed. That is, tension is applied to the four bending wires 33 in a state where the bending portion 12 is made straight without being bent and the screw 34B is screwed fully into the hole of the cylindrical member 34A and made immobile, and all the four bending wires 33 are made taut inside the endoscope 2. At this time, the distance from the proximal end surface 34Bd of the screw 34B (that is, the distal end surface of the crimping member 36) to the proximal end surface 44b of the metal fitting 44 is the same distance LA for all the four bending wires 33.

Note that, as described above, the wire length adjustment member 53 is disposed such that the distance from the distal end surface of the crimping member 36 to the proximal end surface 44b of the metal fitting 44 is the predetermined design dimension LA. Since the distance from the proximal end surface 44b of the metal fitting 44 to a proximal end surface of the coil pipe 45 is constant, the distance from the distal end surface of the crimping member 36 to the proximal end surface of the coil pipe 45 is also set to a predetermined design dimension.

As shown in FIG. 4, the crimping member 36 abuts the proximal end surface 34Bd of the screw 34B as a latch member, and the proximal end portion of the bending wire 33 is latched to the screw 34B by the crimping member 36.

When a bending operation is performed, the suspension frame 31 tilts around the center c of the spherical portion 22, and the crimping member 36 fixed to the bending wire 33 being pulled is pressed against the proximal end surface 34Bd of the screw 34B, but the crimping member 36 fixed to the bending wire 33 which is slackened is not pressed against the proximal end surface 34Bd of the screw 34B.

As described above, when manufacturing the endoscope 2, a worker may adjust the tension of each bending wire 33 simply by placing the bending portion 12 in a straight state where the bending portion 12 is not bent, applying predetermined tension to each bending wire 33, crimping the crimping members 36 abutted against the screws 34B, and cutting the proximal end portion of each of the bending wires 33.

Furthermore, even if the bending wire 33 is twisted, because the bending wire 33 is merely inserted through the hole 34Bb of the screw 34B and is not fixed to the screw 34B, the bending wire 33 is rotatable inside the hole 34Bb of the screw 34B around the axis. Accordingly, even if a force of rotating the bending wire 33 in a direction around the axis is generated as the bending operation is repeatedly performed at the time of use of the endoscope, only the bending wire 33 is rotated around the axis, and the screw 34B is not rotated. Therefore, the bending wire 33 is not slackened due to rotation of the screw 34B.

That is, each screw 34B is fixed to the protruding portion 32, which is a pulling portion, and forms a latch member for latching the proximal end. portion of the bending wire 33 in a state where each bending wire 33 is rotatable in the hole 34Bb of the screw 34B around the axis.

As described above, according to the above embodiment, an endoscope may be provided according to which the number of work steps is reduced, variance in adjustment is reduced, and the bending wire is prevented from being slackened during use of the endoscope.

### (Modification)

The bending wire possibly becomes stretched as the endoscope 2 is used.

Accordingly, with an endoscope of a present modification, spacers are provided between the crimping members 36 and the proximal end surfaces 34Bd of the screws 34B to prevent slacking of the bending wires 33 due to being stretched.

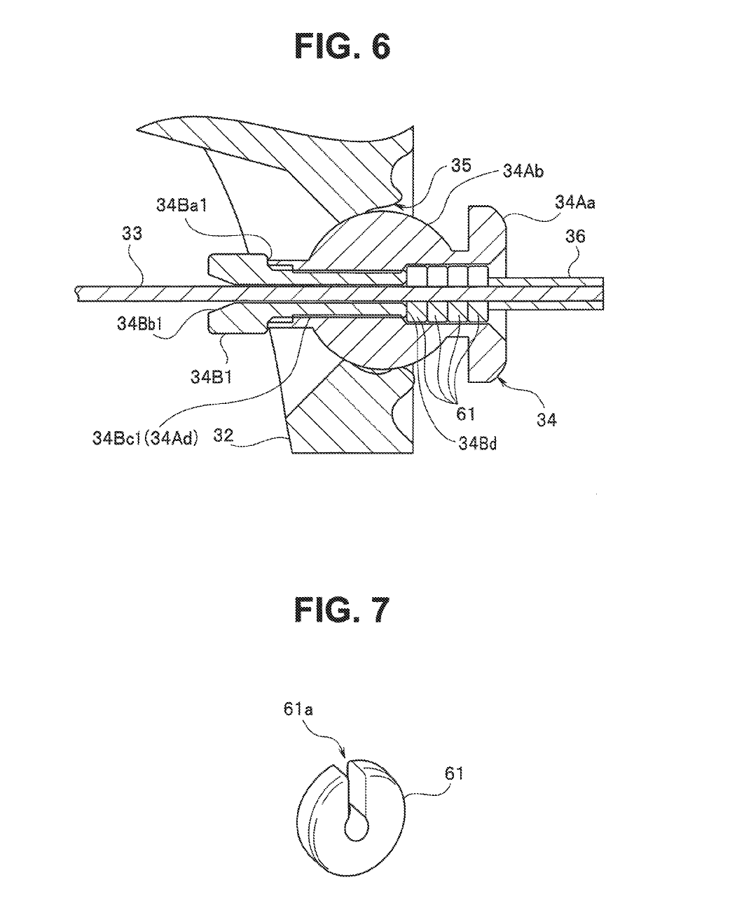

FIG. 6 is a cross-sectional view of the retention portion 35 and the latch portion 34 of the protruding portion 32 provided with a plurality of spacers 61, according to the present modification. FIG. 7 is a perspective view of the spacer 61. Note that, as shown in FIG. 6, a length of a screw 34B1 is shorter than, but may be the same as, a length of the screw 34B in FIG. 4.

As shown in FIG. 6, four spacers 61 for adjusting length are provided between the screw 34B1 and the crimping member 36. As shown in FIG. 7, the spacers 61 are of a material, such as metal or rigid plastic, which is not easily crushed, and each spacer 61 has a disc shape with a cut-out portion 61a so that the bending wire 33 may enter a center of the disc.

The spacers 61 are attached to the bending wire 33 by having the bending wire 33 enter the cut-out portions 61a. The spacers 61 are provided between the crimping member 36 and the proximal end surface 34Bd of the screw 34B1 by being attached to the bending wire 33.

The crimping member 36 is moved toward the proximal end side by the amount of thickness of the spacers 61, and the bending wire 33 is prevented from being slackened due to being stretched.

Note that, because the bending wire 33 may be exposed from between the crimping member 36 and the latch portion 34 by tilting the joystick, the spacers 61 may be attached to the exposed portion of the bending wire 33.

As described above, according to the above embodiment and modification, an endoscope may be provided according to which the number of work steps is reduced, variance in adjustment is reduced, and the bending wire is prevented from being slackened during use of the endoscope.

Because the number of work steps is reduced, the cost is reduced, and because there is not much variance in adjustment among workers, products may be stably manufactured.

The present invention is not limited to the embodiment described above, and various changes and alterations may be made within the scope of the present invention.

What is claimed is:

1. An endoscope comprising:
an insertion section including a bending portion;
a bending wire inserted through the insertion section and configured to bend the bending portion;
a pulling portion configured to pull a proximal end portion of the bending wire; and
a latch member including a screw portion, fixed to the pulling portion by being screwed, and including a hole through which the bending wire is inserted, the latch member being configured to latch the proximal end portion of the bending wire in a state where the bending wire is rotatable in the hole around an axis of the bending wire.

2. The endoscope according to claim 1, wherein the screw portion of the latch member is screwed with a cylindrical member provided in the pulling portion.

3. The endoscope according to claim 2, wherein
the cylindrical member includes a female screw portion, and
the latch member is a screw member having the screw portion, as a male screw portion, formed on an outer circumferential surface.

4. The endoscope according to claim I, comprising a proximal end portion attachment member that is fixed by being attached to the proximal end portion of the bending wire,
wherein the proximal end portion of the bending wire is latched to the latch member by the proximal end portion attachment member abutting against the latch member.

5. The endoscope according to claim 4, comprising a spacer provided between the proximal end portion attachment member and the latch member.

6. The endoscope according to claim 4, wherein the proximal end portion attachment member is a crimping member.

7. The endoscope according to claim 1, wherein the pulling portion is connected to a bending operation member configured to perform a bending operation of the bending portion, and moves according to a movement of the bending operation member.

8. The endoscope according to claim 1, wherein
two bending wires are included as the bending wire in order to bend the bending portion in a first direction and a second direction opposite the first direction, and
the pulling portion moves in such a way that, when one of the two bending wires is pulled, another of the two bending wires is slackened.

9. The endoscope according to claim 1, wherein
four bending wires are included as the bending wire in order to bend the bending portion in a first direction, a second direction opposite the first direction, a third direction orthogonal to the first direction, and a fourth direction opposite the third direction, and
the pulling portion moves in such a way that, when one of a first pair of bending wires for bending in the first and the second directions is pulled, another of the first pair of bending wires is slackened, and when one of a second pair of bending wires for bending in the third and the fourth directions is pulled, another of the second pair of bending wires is slackened.
